(19) United States
(12) Patent Application Publication
(10) Pub. No.: US 2007/0287898 A1
(43) Pub. Date: Dec. 13, 2007

(54) Glove type physiological measuring apparatus

(75) Inventors: Shao-Hung Lee, Taipei (TW); Paul Yang, Taipei (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/449,833

(22) Filed: Jun. 9, 2006

(51) Int. Cl. A61B 5/00

(52) U.S. Cl. 600/323; 600/344

(57) ABSTRACT

A glove type physiological measuring apparatus includes a glove body and an electric connecting base disposed on the glove body and at an area corresponding to the back of a user's hand and electrically coupled to an electric connecting member on a physiological measuring apparatus for measuring various physiological phenomena. The glove body includes an opening disposed at an area corresponding to a finger, an electric connecting base disposed around the periphery of the opening, a biosensor disposed in the opening, and an electric connecting member disposed on the biosensor, and the electric connecting member is electrically connected to the electric connecting base around the opening, and the opening is covered by an adhesive tape for covering and fixing the biosensor, and the biosensor is connected to the physiological measuring apparatus through an electric circuit, so that users can wear more conveniently and clean the apparatus more easily.

### BACKGROUND OF THE INVENTION

1. Field of the Invention

The present invention relates to a physiological measuring apparatus, and more particularly to a glove type physiological measuring apparatus that integrates a physiological measuring apparatus, a detector, and a glove body and also provides a convenient way for users to wear and wash the apparatus.

2. Description of the Related Art

As our life quality is improved and more attentions are paid to our health, people use various different measuring apparatuses and materials to examine their physical conditions, and then make appropriate adjustments and take good care of their physical situations. One of the common check-up items is the measurement of heartbeat, and a traditional wearing type heartbeat measuring apparatus as shown in FIG. 1 has a glove 100, and the glove 100 has a fixed measuring apparatus 110 for measuring a heartbeat, and the measuring apparatus 110 has a display screen 111 for displaying a heartbeat value. The measuring apparatus 110 further has an electric wire 112, and an end of the electric wire 112 is connected to a light detector 130, and an end of the glove 100 is extended into an area corresponding to a finger, and the area has a finger sheath 120. When the heartbeat measuring apparatus 110 is used, a user has to wear the glove 100 onto a user's palm first, and then the finger sheath 120 is sheathed onto a user's finger, and the light detector 130 is stuffed into the finger sheath 120. By the refraction of a light, our heartbeat can be detected. Since it is necessary to stuff the light detector 130 into the finger sheath 120 for the measurement, and thus making the wearing and the measurement inconvenient. Further, the measuring apparatus 110 is fixed onto the surface of the glove 100, and thus it is difficult to clean the measuring apparatus 110.

### SUMMARY OF THE INVENTION

In view of the foregoing shortcomings of the prior art apparatuses, the inventor of the present invention based on years of experience in the related field to conduct extensive researches and experiments, and finally developed a glove type physiological measuring apparatus in accordance with the present invention.

The primary objective of the present invention is to provide a glove type physiological measuring apparatus, and the apparatus has a glove body, and the glove body has an electric connecting base disposed at an area corresponding to the back of a user's hand, and the electric connecting base is electrically connected to an electric connecting member on a physiological measuring apparatus. The physiological measuring apparatus is used for measuring various physiological phenomena of a human body. Further, the glove body has an opening disposed at an area corresponding to a finger, and the periphery of the opening has an electric connecting base, and the opening installs a biosensor therein. The biosensor has an electric connecting member electrically connected to an electric connecting base disposed around the opening. The opening includes an adhesive tape for covering and fixing the biosensor. Further, the biosensor is electrically connected to the physiological measuring apparatus through an electric circuit, so that users can wear the apparatus more conveniently and clean the apparatus more easily.

Another objective of the present invention is to provide a glove type physiological measuring apparatus, and the apparatus has a glove body, and the glove body has an electric connecting base disposed at an area corresponding to the back of a hand, and the adhering member is adhered with a physiological measuring apparatus, and the physiological measuring apparatus includes an adhering body, such that the physiological measuring apparatus can be adhered with the adhering member by the adhering body and fixed onto the surface of the glove.

A further objective of the present invention is to provide a physiological measuring apparatus, wherein the glove body has an opening disposed at an area corresponding to a finger, and the openings separately contain a light transmitter and a light receiver for measuring the concentration of blood oxygen.

Another further objective of the present invention is to provide a physiological measuring apparatus, and the glove body includes an electric circuit disposed between the physiological measuring apparatus and the biosensor, and the electric circuit woven together with the fibers of the glove body.

To achieve the foregoing objectives of the present invention, the technical measures and its performance will now be described in more detail hereinafter with reference to the accompanying drawings that show various embodiments of the invention.

### BRIEF DESCRIPTION OF THE DRAWINGS

FIG. 1 is a schematic view of a prior art apparatus;

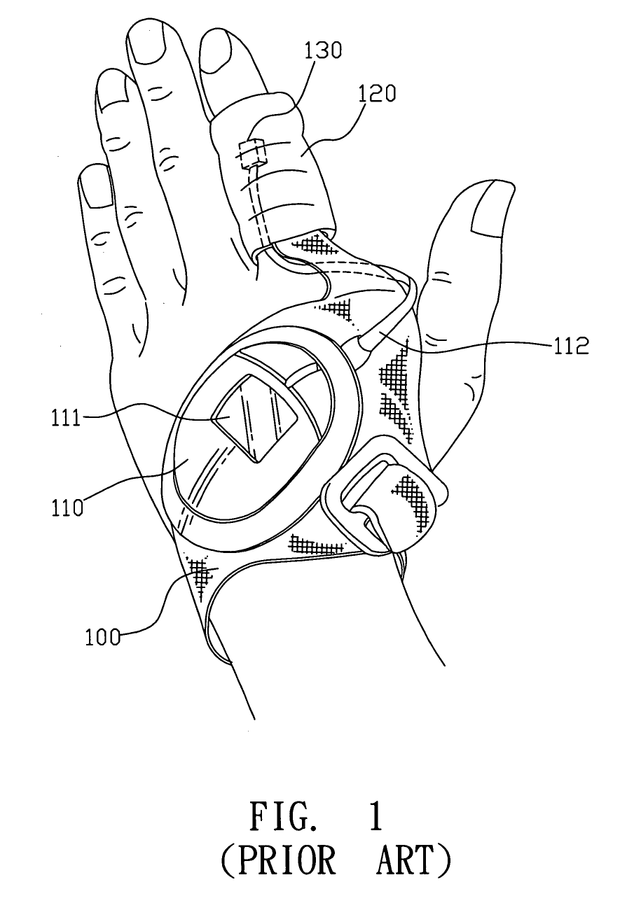

FIG. 2 is an exploded view of the present invention;

FIG. 3 is a perspective view of a preferred embodiment of the present invention;

FIG. 4 is a perspective view of another preferred embodiment of the present invention; and

FIG. 5 is a schematic view of a physiological measuring apparatus being adhered onto a glove according to the present invention.

### DETAILED DESCRIPTION OF THE PREFERRED EMBODIMENTS

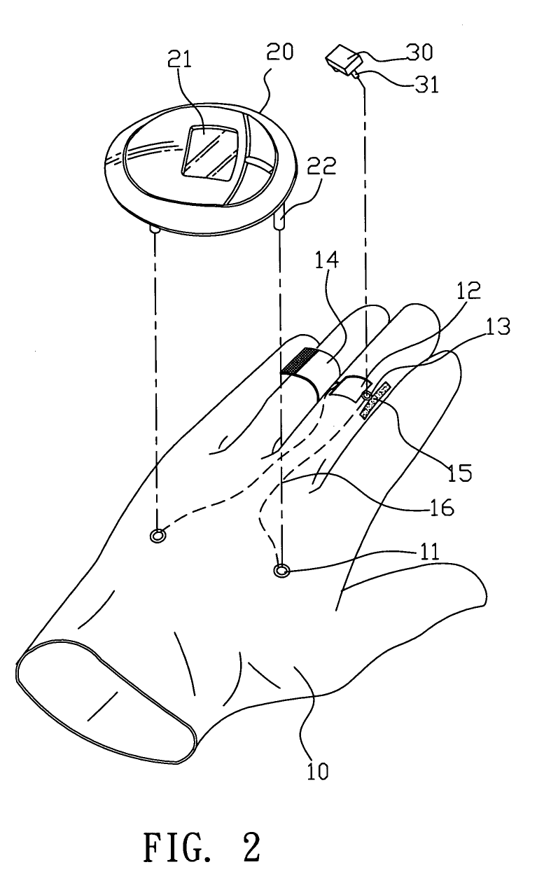

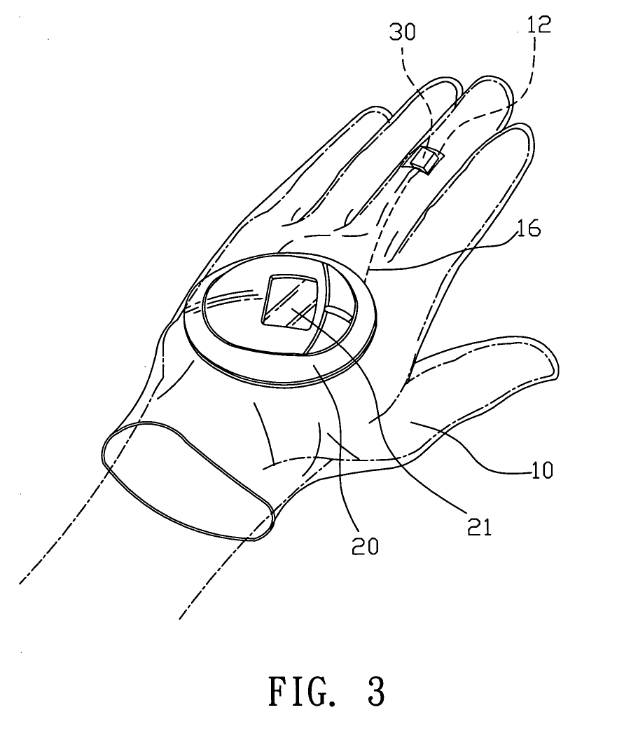

Referring to FIGS. 2 and 3 for a glove type physiological measuring apparatus of the present invention, the apparatus includes a glove body 10 made of any material and worn on a user's hand, and the glove body 10 has an electric connecting base 11 disposed on an area corresponding to the back of the user's hand and a physiological measuring apparatus 20 connected to the glove body 10. The physiological measuring apparatus 20 is used for measuring various physiological phenomena of a human body, and the surface of the physiological measuring apparatus 20 includes a display screen 21 for displaying the measured values, and an electric connecting member 22 disposed on the physiological measuring apparatus 20 is inserted into the electric connecting base 11 for an electric connection. Further, the glove body 10 has an opening 12 disposed at an area corresponding to any finger (which is the index finger for this embodiment). The opening 12 includes an electric connecting base 13 disposed at its periphery and an adhesive tape 14 disposed on a side of the electric connecting base 13, and another end of the adhesive tape 14 is adhered onto an adhering area 15 at another side of the electric connecting base 13. Further, the opening 12 installs a biosensor 30 therein (this embodiment adopts a light detector for detecting the pulse of a human body), and the biosensor 30 includes an electric connecting member 31 thereon, and the electric connecting member 31 is inserted into the electric connecting base 13 around the periphery of the opening 12 for an electric connection. An adhesive tape 14 is covered onto the opening 12 for covering and fixing the biosensor 30. Further, the biosensor 30 is electrically coupled to the physiological measuring apparatus 20 through an electric circuit 16 woven in the glove body 10, so that a user can wear the apparatus more conveniently and clean the apparatus more easily.

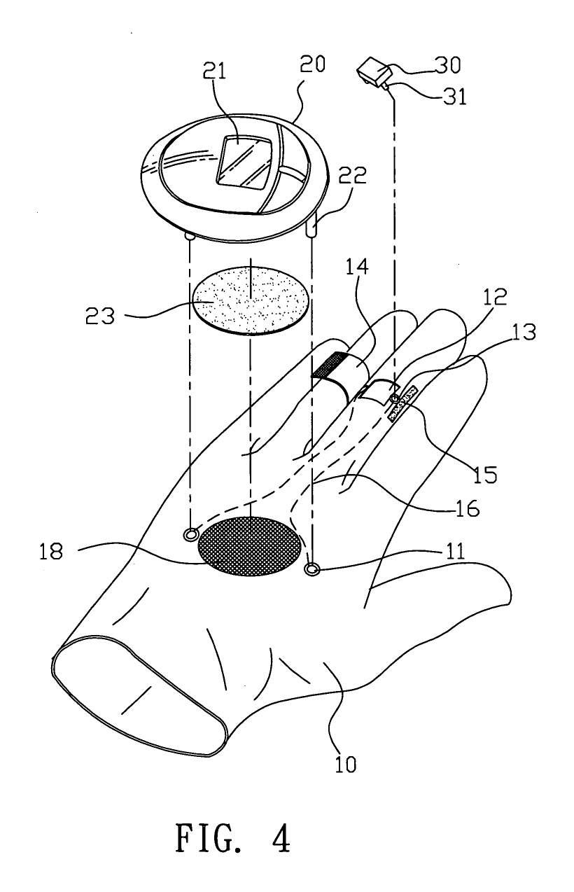

Referring to FIG. 4, an adhering member 18 is disposed on the glove body 10 at an area corresponding to a palm (in this embodiment, a sticker or an adhesive tape is fixed onto the back of a hand). Further, the physiological measuring apparatus 20 includes an adhering body 23 disposed thereunder (a sticker or an adhesive tape is used in this embodiment), such that the physiological measuring apparatus 20 can be attached onto the adhering member 18 through the adhering body 23 and fixed onto the surface of the glove body 10.

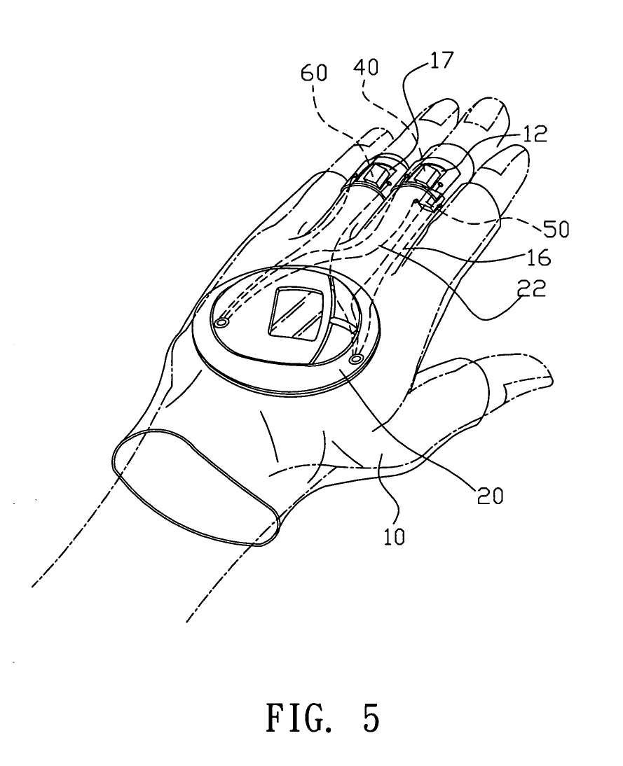

Referring to FIG. 5 for s second preferred embodiment of the present invention, the glove body 10 includes an opening 12 disposed at an area corresponding to a finger, and the openings 12 separately contain a light transmitter 40 and a light receiver 50 coupled to the physiological measuring apparatus 20 for measuring the concentration of blood oxygen. Alternatively, an opening 17 corresponding to a finger (which is a ring finger in this embodiment) other than the one corresponding to the opening 12 of the glove body 10 is disposed at an area corresponding to that finger. The biosensor 60 installed in the opening 17 is used for measuring the physiological phenomena of a human body, and the biosensor 60 can work together with the physiological measuring apparatus 20 for measuring a body temperature and the like.

In summation of the description above, the glove type physiological measuring apparatus of the invention features a simple structure and a high precision, so that users can examine their physical conditions anytime, and then make appropriate adjustments to take good care of their health conditions. The present invention provides a feasible solution, and a patent application is duly filed accordingly.

However, it is to be noted that the preferred embodiments disclosed in the specification and the accompanying drawings are not intended to limit the invention. To the contrary, it is intended to cover various modifications and similar arrangements and procedures, and thus the scope of the appended claims should be accorded the broadest interpretation so as to encompass all such modifications and similar arrangements and procedures.

What is claimed is:

1. A glove type physiological measuring apparatus, comprising:
a glove body, being worn on a user's palm and including an electric connecting base separately disposed on said glove body and at a position corresponding to the back of a hand and a finger, an electric circuit installed between said electric connecting bases at the back of said hand and said finger, and at least one opening disposed on said glove body and at an area corresponding to said finger;
a physiological measuring apparatus, including an electric connecting member mounted onto said electric connecting base and disposed at the position of the back of said hand of said glove body;
a biosensor, including an electric connecting member mounted onto said electric connecting base at the position of said finger of said glove body and coupled to said physiological measuring apparatus through said electric circuit, and said biosensor being installed in an opening;
thereby, a physiological measurement can be made at the position of said biosensor disposed on said opening.

2. The glove type physiological measuring apparatus of claim 1, wherein said glove body includes an opening disposed at an area corresponding to said finger for installing said biosensor with different functions.

3. The glove type physiological measuring apparatus of claim 2, wherein said biosensor comprises a light transmitter and a light receiver for measuring the concentration of blood oxygen.

4. The glove type physiological measuring apparatus of claim 1, wherein said glove body includes an adhering member disposed thereon, and said physiological measuring apparatus includes an adhering body disposed thereunder, and said adhering body is adhered to said adhering member.

5. The glove type physiological measuring apparatus of claim 1, wherein said opening of said glove body is covered by an adhesive tape for fixing said
